(19) United States
(12) Patent Application Publication
(10) Pub. No.: US 2022/0023584 A1
(43) Pub. Date: Jan. 27, 2022

(54) ARTIFICIAL INTELLIGENCE-BASED NON-INVASIVE NEURAL CIRCUIT CONTROL TREATMENT SYSTEM AND METHOD FOR IMPROVING SLEEP

(71) Applicant: EWHA UNIVERSITY - INDUSTRY COLLABORATION FOUNDATION, Seoul (KR)

(72) Inventors: Hyang Woon LEE, Gangnam-gu, Seoul (KR); Je Won KANG, Mapo-gu, Seoul (KR); Jung Rok LEE, Seodaemun-gu, Seoul (KR); Sang Beom JUN, Seocho-gu, Seoul (KR); Chang Hyeon JI, Seocho-gu, Seoul (KR)

(21) Appl. No.: 17/311,244

(22) PCT Filed: Nov. 5, 2019
(86) PCT No.: PCT/KR2019/014901

§ 371 Date: Jul. 29, 2021

(51) Int. Cl. A61M 21/02; G16H 20/70; G16H 40/67

(52) U.S. Cl. CPC A61M 21/02; A61M 2021/0022; G16H 40/67; G16H 20/70

(57) ABSTRACT

Provided is an artificial intelligence-based noninvasive brain circuit control therapy system for sleep enhancement, the system including a wearable device including a first wearable member and a second wearable member formed to be wearable on a body of a user, a first sensor unit disposed on the first wearable member to detect an electroencephalogram (EEG), a second sensor unit disposed on the second wearable member to detect a biometric signal different from the EEG, and a stimulation means disposed on the first wearable member to stimulate the brain according to a stimulation signal provided thereto; a learning unit configured to machine-learn a criterion for determination of a sleep stage of the user based on a first sensing signal generated by the first sensor unit and a second sensing signal generated by the second sensor unit; and a determination unit configured to determine a current sleep stage of the user based on the criterion for determination, generate a stimulation signal corresponding to a determined sleep stage, and provide the stimulation signal to the stimulation means.

### TECHNICAL FIELD

The present disclosure relate to an artificial intelligence-based noninvasive brain circuit control therapy system for sleep enhancement and method therefor.

### BACKGROUND ART

A brain wave and an electrocardiogram are used as indicators to evaluate brain activity. A brain wave, that is, an electroencephalogram (EEG) is a test method capable of evaluating cerebral function. An EEG may indicate, for example, whether brain functions, especially brain activity, are weakening or increasing. Therefore, the value of an EEG is recognized as being able to grasp spatial and temporal fluctuations in brain activity that change from moment to moment.

Electrical activities of the brain reflected in an EEG is determined by neurons, gila cells, and a blood-brain barrier, and it is known that electrical activities are mainly generated by neurons. The gila cells, which account for half of the brain's weight, control the flow of ions and molecules at synapses, which are areas interconnecting neurons, and support, maintain, and repair structures between neurons. The blood-brain barrier selectively transmits therethrough only necessary substances from among various substances in the blood vessels of the brain. Changes in brain waves due to the gila cells and the blood-brain barrier occur little by little and slowly, whereas changes in brain waves due to neuronal activity are significant, rapid, and diverse.

On the other hand, sleep is known to consolidate memories. Slow oscillations of the cerebral cortex (predominantly with frequencies below 1 Hz), thalamo-cortical spindles (with predominantly frequencies from 7 to 15 Hz), and sharp-wave ripples in the hippocam pus (predominantly with frequency from 100 Hz to 250 Hz) represent the basic rhythm of a slow wave sleep state, and all these rhythms are known to be related to the consolidation of hippocampal-dependent memory during sleep.

### DESCRIPTION OF EMBODIMENTS

### Technical Problem

Embodiments of the present disclosure provide an artificial intelligence-based noninvasive brain circuit control therapy system for sleep enhancement that determines wake and sleep stages through machine learning by measuring multi-biometric signals like brain waves, heartbeat, eye movement, and muscle activity and control a sleep stage by stimulating sleep controlling brain regions by using a transcranial noninvasive neuromodulatory device rather than an implantable electrode, thereby enhancing the cognitive-emotional function.

### Solution to Problem

According to an aspect of the present disclosure, an artificial intelligence-based noninvasive brain circuit control therapy system for sleep enhancement, the system includes a wearable device including a first wearable member and a second wearable member formed to be wearable on a body of a user, a first sensor unit disposed on the first wearable member to detect an electroencephalogram (EEG), a second sensor unit disposed on the second wearable member to detect a biometric signal different from the EEG, and a stimulation means disposed on the first wearable member to stimulate the brain according to a stimulation signal provided thereto; a learning unit configured to machine-learn a criterion for determination of a sleep stage of the user based on a first sensing signal generated by the first sensor unit and a second sensing signal generated by the second sensor unit; and a determination unit configured to determine a current sleep stage of the user based on the criterion for determination, generate a stimulation signal corresponding to a determined sleep stage, and provide the stimulation signal to the stimulation means.

### Advantageous Effects of Disclosure

An artificial intelligence-based noninvasive brain circuit control therapy system for sleep enhancement according to embodiments of the present disclosure measures multi-biometric signals in real time, analyzes a sleep stage through an artificial intelligence, and performs noninvasive local brain stimulation therapy in core sleep controlling brain circuit target regions, thereby enhancing sleep and cognitive brain functions.

### BRIEF DESCRIPTION OF DRAWINGS

FIG. 1 is a diagram showing an example of a network environment according to an embodiment of the present disclosure.

FIG. 2 is a conceptual diagram for describing a brain circuit that controls sleep-wake and cognitive-emotional brain functions.

FIG. 3 is a conceptual diagram for describing a structure of sleep overnight.

FIG. 4 is a schematic block diagram showing an artificial intelligence-based noninvasive brain circuit control therapy system for sleep enhancement according to an embodiment of the present disclosure.

FIG. 5 is a diagram for describing the artificial intelligence-based noninvasive brain circuit control therapy system for sleep enhancement according to an embodiment of the present disclosure.

FIG. 6 is a diagram showing a structure for determining a sleep stage and controlling ultrasound stimulation in an artificial intelligence-based noninvasive brain circuit control therapy system for sleep enhancement according to an embodiment of the present disclosure.

FIG. 7 is a diagram for describing a noise remover and signal quality enhancer using a convolutional neural network (CNN).

FIG. 8 is a diagram for describing a sleep stage determination algorithm.

FIG. 9 is a diagram showing major parts of the human brain for controlling sleep.

FIG. 10 is a diagram showing a time distribution of each stage of REM sleep and NREM sleep during overnight sleep and a correlation between sleep spindles and slow wave and high frequency EEG.

### BEST MODE

According to an aspect of the present disclosure, an artificial intelligence-based noninvasive brain circuit control therapy system for sleep enhancement, the system includes a wearable device including a first wearable member and a second wearable member formed to be wearable on a body of a user, a first sensor unit disposed on the first wearable member to detect an electroencephalogram (EEG), a second sensor unit disposed on the second wearable member to detect a biometric signal different from the EEG, and a stimulation means disposed on the first wearable member to stimulate the brain according to a stimulation signal provided thereto; a learning unit configured to machine-learn a criterion for determination of a sleep stage of the user based on a first sensing signal generated by the first sensor unit and a second sensing signal generated by the second sensor unit; and a determination unit configured to determine a current sleep stage of the user based on the criterion for determination, generate a stimulation signal corresponding to a determined sleep stage, and provide the stimulation signal to the stimulation means.

In an embodiment of the present disclosure, the second sensor unit may detect an electrooculogram (EOG) and generates the second sensing signal, and the second wearable member may be connected to the first wearable member and be wearable on the head of the user.

In an embodiment of the present disclosure, the second sensor unit may detect an electromyogram (EMG) and generates the second sensing signal, and the second wearable member may be wearable on a wrist of the user or may be connected to the first wearable member and wearable on the face of the user.

In an embodiment of the present disclosure, the second sensor unit may detect a photoplethysmogram (PPG) and generates the second sensing signal, and the second wearable member may be wearable on the chest or a finger of the user or may be connected to the first wearable member and wearable on an ear of the user.

In an embodiment of the present disclosure, the second sensor unit may generate the second sensing signal by sensing an EOG, an EMG, and a PPG, and the second wearable member may include a wearable part 2-1 that is connected to the first wearable member and is wearable on the head of the user, a wearable part 2-2 that is wearable on a wrist of the user, and a wearable part 2-3 that is wearable on the chest of the user.

In an embodiment of the present disclosure, the stimulation means may be an ultrasound generating means for generating ultrasound stimulation.

In an embodiment of the present disclosure, the first sensor unit may generate the first sensing signal by sensing the EEG in the time series order, and the second sensor unit may generate the second sensing signal synchronized with the first sensing signal by detecting the other biometric signal in time series order.

In an embodiment of the present disclosure, the learning unit may extract a first feature from the first sensing signal generated in the time series order, extract a second feature from the second sensing signal generated in the time series order, and learn the criterion for determination based on the first feature and the second feature including temporal information.

According to another aspect of the present disclosure, an artificial intelligence-based noninvasive brain circuit control therapy method for sleep enhancement, the method includes receiving a first sensing signal generated by a first sensor unit that detects an electroencephalogram (EEG); receiving a second sensing signal generated by a second sensor unit that detects a biometric signal other than the EEG; and machine-learning a criterion for determination of a sleep stage of a user based on the first sensing signal and the second sensing signal.

In an embodiment of the present disclosure, the first sensor unit may generate the first sensing signal by sensing the EEG in the time series order, and the second sensor unit may generate the second sensing signal synchronized with the first sensing signal by detecting the other biometric signal in time series order.

In an embodiment of the present disclosure, the machine-learning of the criterion for determination may include extracting a first feature from the first sensing signal generated in the time series order; extracting a second feature from the second sensing signal generated in the time series order; and learning the criterion for determination based on the first feature and the second feature including temporal information.

In an embodiment of the present disclosure, the extracting of the first feature and the extracting of the second feature may be performed noninvasively.

In an embodiment of the present disclosure, the method may further include determining a current sleep stage of the user based on the criterion for determination; and generating a stimulation signal corresponding to a determined sleep stage and providing the stimulation signal to a stimulation means.

In an embodiment of the present disclosure, the second sensor unit may detect an electrooculogram (EOG) and generate the second sensing signal.

In an embodiment of the present disclosure, the second sensor unit may detect an electromyogram (EMG) and generate the second sensing signal.

In an embodiment of the present disclosure, the second sensor unit may detect a photoplethysmogram (PPG) and generate the second sensing signal.

In an embodiment of the present disclosure, the second sensor unit may detect an EOG, an EMG, and a PPG to generate the second sensing signal.

According to another aspect of the present disclosure, there is provided a computer program stored in a medium for executing the above-stated method by using a computer.

Other aspects, features, and advantages will become apparent from the following drawings, claims, and detailed description of the present disclosure.

### MODE OF DISCLOSURE

The present disclosure may include various embodiments and modifications, and embodiments thereof will be illustrated in the drawings and will be described herein in detail. The effects and features of the present disclosure and the accompanying methods thereof will become apparent from the following description of the embodiments, taken in conjunction with the accompanying drawings. However, the present disclosure is not limited to the embodiments described below, and may be embodied in various modes.

Reference will now be made in detail to embodiments, examples of which are illustrated in the accompanying drawings. In the drawings, the same elements are denoted by the same reference numerals, and a repeated explanation thereof will not be given.

It will be understood that although the terms “first”, “second”, etc. may be used herein to describe various elements, these elements should not be limited by these terms. These elements are only used to distinguish one element from another.

As used herein, the singular forms “a,” “an” and “the” are intended to include the plural forms as well, unless the context clearly indicates otherwise.

It will be further understood that the terms “comprises” and/or “comprising” used herein specify the presence of stated features or components, but do not preclude the presence or addition of one or more other features or components.

It will be understood that when a layer, region, or component is referred to as being “formed on” another layer, region, or component, it can be directly or indirectly formed on the other layer, region, or component. That is, for example, intervening layers, regions, or components may be present.

Sizes of elements in the drawings may be exaggerated for convenience of explanation. In other words, since sizes and thicknesses of components in the drawings are arbitrarily illustrated for convenience of explanation, the following embodiments are not limited thereto.

When a certain embodiment may be implemented differently, a specific process order may be performed differently from the described order. For example, two consecutively described processes may be performed substantially at the same time or performed in an order opposite to the described order.

In the specification, when a film, region, or component is connected to another film, region, or component, the films, regions, or components may not only be directly connected, but may also be indirectly connected via an intervening film, region, or component therebetween. For example, when a film, region, component is electrically connected to another film, region, or component, the films, regions, or components may not only be directly electrically connected, but may also be indirectly electrically connected via an intervening film, region, or component therebetween.

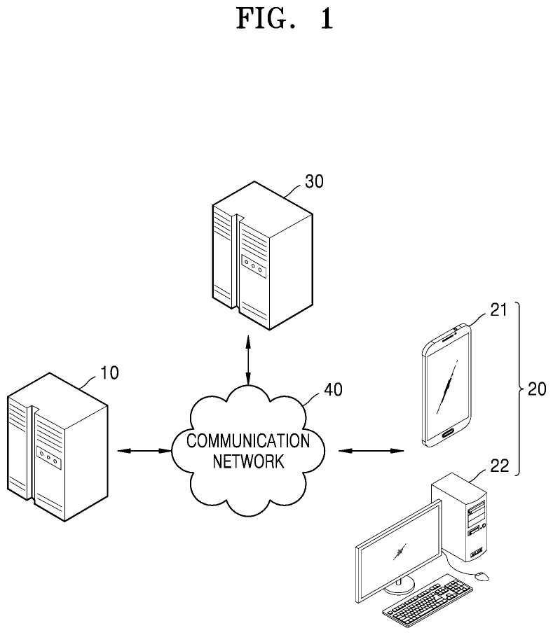

FIG. 1 is a diagram showing an example of a network environment according to an embodiment of the present disclosure.

FIG. 1 shows an example in which the network environment includes a user terminal 20, a server 10, an external device 30, and a communication network 40. However, the network environment shown in FIG. 1 is merely an example for explanation of the present disclosure, and the number of user terminals and the number of servers is not limited to those shown as in FIG. 1.

In an artificial intelligence-based noninvasive brain circuit control therapy system for sleep enhancement according to an embodiment of the present disclosure, the server 10 may receive a multi-biometric signal including an electroencephalogram (EEG) signal sensed by the external device 30, determine a sleep stage, detect a spindle signal, generate a stimulation signal to stimulate a sleep-controlling brain region, transmit the generated stimulation signal to the external device 30 or a separate external device to stimulate the brain of a user, thereby controlling the sleep stage and improving cognitive-emotional function.

The user terminal 20 may be a stationary terminal 22 or a mobile terminal 21 implemented as a computer device. The user terminal 20 may be a terminal for transmitting data received from a wearable device 110 to be described later to the server 10 and the external device 30. Examples of the user terminal 20 include a smart phone, a mobile phone, a navigation system, a computer, a laptop computer, a digital broadcasting terminal, a personal digital assistants (PDA), a portable multimedia player (PMP), a tablet PC, etc. For example, a user terminal 121 may communicate with another user terminal 22 and/or the servers 10 and 30 through the communication network 40 via a wireless communication method or a wire.

The external device 30 may refer to various devices that transmit and receive data to and from the server 10 and the user terminal 20 through the communication network 40. In detail, in the present disclosure, the external device 30 may be a measuring device capable of measuring multi-biometric signals, such as an EEG or a photoplethysmogram (PPG) of a user, or may be a stimulation device that transmits a stimulation signal to a sleep-controlling brain region of the user. The external device 30 may be a wearable device capable of measuring an EEG or transmitting a stimulation signal by being worn by a user while the user is sleeping, but is not necessarily limited thereto. Meanwhile, the multi-biometric signal detected by the external device 30 may be a signal, such as an EEG, a PPG, an eye movement signal, or an electromyogram (EMG).

In this specification, the external device 30 may transmit and receive data directly to and from the server 10 through the communication network 40. However, when data is transmitted to the user terminal 20 by using a short-range communication network, the user terminal 20 may transmit the data to the server 10 through the communication network 40 or transmit data to the server 10 after a necessary data processing through a pre-set algorithm. Also, the user terminal 20 may perform a function of notifying information including a determined sleep stage to a user. However, the present disclosure is not limited thereto, and the user terminal 20 may perform the function of the server 10 by storing data in the user terminal 20 without transferring the data to the server 10.

A communication protocol is not limited and may include not only communication protocols that utilizes communication networks that may be included in the communication network 40 (e.g., mobile communication networks, wired Internet, wireless Internet, and broadcasting networks), but also short-range wireless communications between devices. For example, the communication network 40 may include any one or more networks from among networks including a personal area network (PAN), a local area network (LAN), a campus area network (CAN), a metropolitan area network (MAN), a wide area network (WAN), a broadband network (BBN), the Internet, etc. Also, the communication network 40 may include any one or more from among network topologies including a bus network, a star network, a ring network, a mesh network, a star-bus network, a tree or a hierarchical network, etc., but is not limited thereto.

The server 10 may be implemented by a computer device or a plurality of computer devices that communicates with the user terminal 20 through the communication network 40 to provide commands, codes, files, contents, services, etc.

For example, the server 10 may provide a file for installation of an application to the user terminal 121 connected through the communication network 40. In this case, the user terminal 121 may install an application by using a file provided from the server 10. Also, a service or content provided by the server 10 may be received by accessing the server 10 under the control of an operating system (OS) and at least one program (e.g., a browser or an installed application) included in the user terminal 121. In another example, the server 10 may establish a communication session for data transmission/reception and route data transmission/reception between user terminals 20 through the established communication session.

When a first sensing signal S1 and a second sensing signal S2, which are multi-biometric signals, the server 10 according to an embodiment of the present disclosure may learn a criterion for determining a sleep stage of a user based on deep learning based on the first sensing signal S1 and the second sensing signal S2, generate a stimulation signal corresponding to a determined sleep stage, and provide the stimulation signal as a stimulation means. In another embodiment, the server 10 may perform a function of learning a criterion for determination based on deep learning and transmit the criterion for determination to the external device 30, and thus the external device 30 may determine a sleep stage and generate a stimulation signal. However, the present disclosure is not limited thereto, and the function of learning a criterion for determination may be performed even by the user terminal 20 having a processor. In this case, the user terminal 20 may learn the criterion for determination by itself without accessing the server 10 and may generate a user-customized criterion for determination through deep learning.

Hereinafter, a brain circuit that controls human sleep-wake and cognitive-emotional brain functions will be first described, and then, an artificial intelligence-based noninvasive brain circuit control therapy system 100 for sleep enhancement according to an embodiment of the present disclosure will be described.

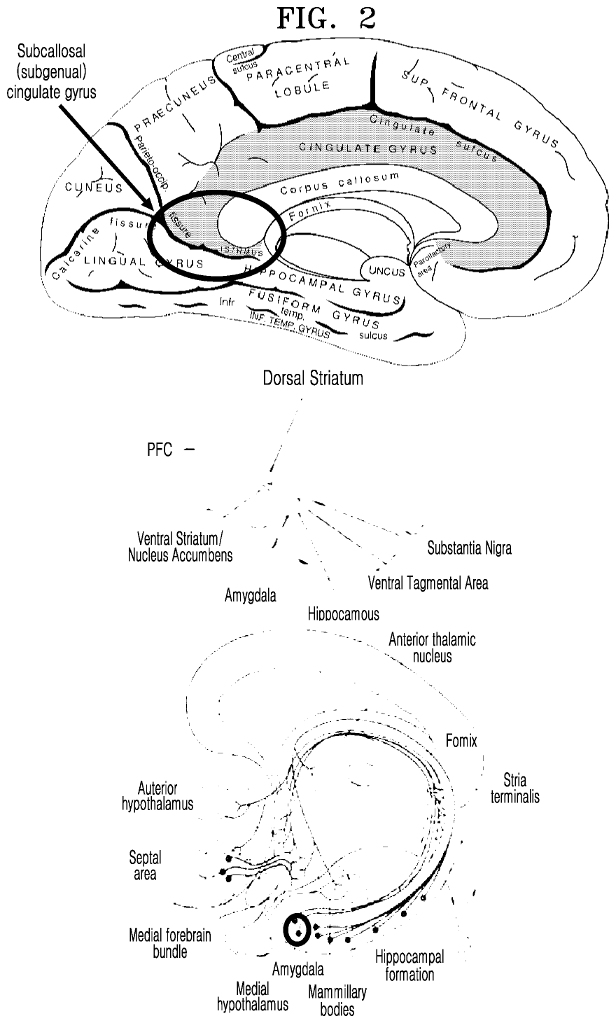

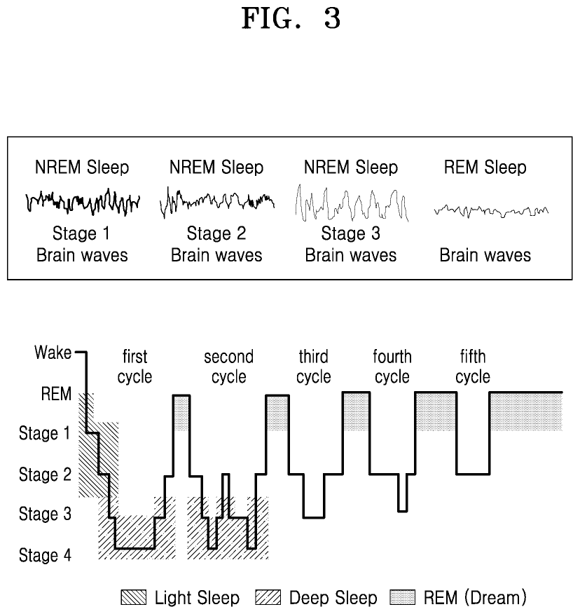

FIG. 2 is a conceptual diagram for describing a brain circuit that controls sleep-wake and cognitive-emotional brain functions, and FIG. 3 is a conceptual diagram for describing a structure of sleep overnight.

Sleep plays an important role in consolidation necessary for memory and learning. When the sleep-wake circadian rhythm is disturbed due to a sleep disorder, it may lead to sleep deprivation and daytime sleepiness, and the vicious cycle of worsening sleep problems by aggravating work/environmental/psychological stress is repeated. As a result, it is difficult to exert maximum cognitive ability, which may impede learning and brain function development.

Currently, in the case of depression, non-invasive brain stimulation, particularly repetitive transcranial magnetic stimulation, has already been approved by the FDA for use in the treatment of drug-refractory mild depressive disorder in the United States in 2007, and. in Korea, has been approved by the Ministry of Food and Drug Safety in 2014 and is being used. However, in the case of sleep disorders, especially insomnia, although restless legs syndrome, narcolepsy, and obstructive sleep apnea are known to cause cognitive and emotional abnormalities, the reality is that there is no alternative other than cognitive behavioral therapy for insomnia, drug therapy for restless legs syndrome and narcolepsy, and positive pressure breathing therapy for obstructive sleep apnea.

Recently, a small number of researchers have published results on treatments of sleep disorders caused by repetitive transcranial magnetic stimulation, but there are few studies on the core brain circuits of sleep disorders that cause emotional/cognitive brain dysfunction and the discovery of brain circuits related to the improvement of sleep disorders and emotional/cognitive brain function abnormalities by non-invasive stimulations on the core brain circuits.

Therefore, the present disclosure relates to a system for discovering core human brain circuits related to sleep improvement through non-invasive local brain stimulation and applying them to the human body for establishing clinical research protocols and sleep improvement services through extended application of general population and patients with various sleep disorders.

Referring to FIG. 2, brain circuits that control sleep-wake and cognitive-emotional brain functions are mainly deep portions of the brain like a thalamus, a basal forebrain, and a brainstem, wherein cerebral cortexes and subcortical brain regions like a prefrontal cortex, an amygdala of a limbic system, a cingulated cortex, and a hippocam pus, which are involved in the regulation of emotional and cognitive functions, are closely linked structurally and functionally. A network of brain regions that influences one another in relation to sleep-wake control may be considered as a core sleep brain circuit, and a brain connectivity analysis may be applied to EEG data from the conventional standard polysomnography test to find the network.

Meanwhile, referring to FIG. 3, human sleep may be basically divided into non-rapid eye movement (NREM) sleep and rapid eye movement (REM) sleep showing rapid eye movement. The NREM sleep may be divided into N1 sleep (stage 1), N2 sleep (stage 2), and N3 sleep (stage 3) according to the depth of sleep. The higher the stage of sleep is, the stronger the stimulation needs to be to switch to the wake state. The purpose of the present disclosure is, by applying appropriate ultrasonic stimulation according to a sleep phase, to induce effective sleep initiation and emotional relaxation in the wake state or to strengthen hippocampal memory during slow wave sleep. In the present specification, in order to determine a sleep stage, sleep spindles and slow wave sleep may be used as measured EEG indicators.

Here, sleep spindles are bursts of neural oscillatory activity with a frequency from 10 Hz to 16 Hz lasting for at least 0.5 seconds caused by the interaction of a thalamic reticular nucleus (TRN) with other thalamic nuclei during a second stage of the NREM sleep. Sleep spindles are observed in the NREM sleep of mammals, and their functions are known to govern both sensory processing and long term memory consolidation, and the formation of spindles is known as a waveform generated when a signal is transmitted from one part of the cerebral cortex to another.

The slow wave sleep is the deepest sleep stage 3 in the NREM sleep, features the delta wave, which is a large waveform in EEG, and is an important stage for memory consolidation. According to the criteria for judging the sleep stage of the American Academy of Sleep Medicine (AASM), which was revised in 2008, when delta waves with a frequency from 0.5 Hz to 2.0 Hz of a tall 75-microvolt are observed per 30 seconds or more per epoch, it is read as slow wave sleep and is usually observed for the longest duration during the first two sleep cycles of the first 3 hours of one-night sleep. It is known that the slow wave sleep is important for memory consolidation for converting various information collected during the day into long-term memory.

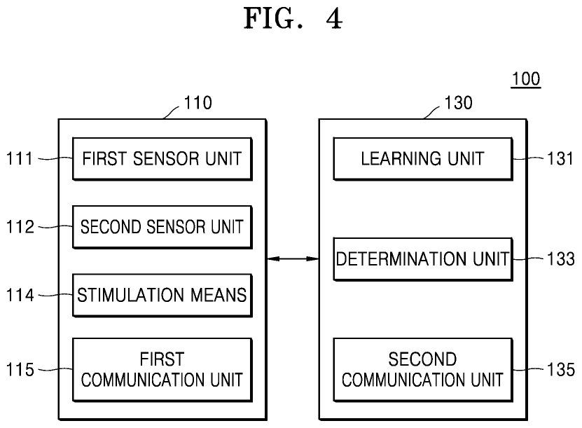

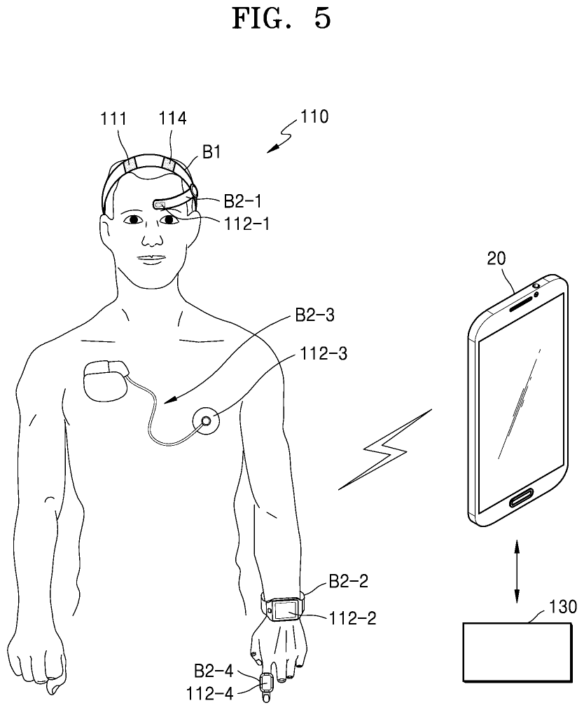

FIG. 4 is a schematic block diagram showing an artificial intelligence-based noninvasive brain circuit control therapy system for sleep enhancement 100 according to an embodiment of the present disclosure, and FIG. 5 is a diagram for describing the artificial intelligence-based noninvasive brain circuit control therapy system 100 for sleep enhancement according to an embodiment of the present disclosure.

Referring to FIGS. 4 and 5, the artificial intelligence-based noninvasive brain circuit control therapy system 100 for sleep enhancement according to an embodiment of the present disclosure includes a wearable device 110 and a server unit 130, which includes a learning unit 131 and a determination unit 133.

Here, the wearable device 110 may correspond to the external device 30 of FIG. 1, and the server unit 130 may correspond to the server 10 of FIG. 1. Although FIG. 4 shows that the wearable device 110 communicates directly with the server unit 130, the present disclosure is not limited thereto, and the wearable device 110 and the server unit 130 may transmit and receive data via the user terminal 20 as shown in FIG. 5.

The wearable device 110 may include a first wearable member B1, a second wearable member B2, a first sensor unit 111, a second sensor unit 112, a stimulation means 114, and a first communication unit 115.

The first wearable member B1 may be formed to be wearable on a user's body. As shown in FIG. 5, the first wearable member B1 may be a member in the form of a headband, a helmet, or a band to be worn on the head of a user.

The first sensor unit 111 may be provided at the first wearable member B1 and may generate a first sensing signal S1 by sensing an electroencephalogram (EEG). The first sensor unit 111 may include one or more measuring electrodes, and the measuring electrodes may be arranged at locations at which signal detection is not limited by hairs instead of being worn on the entire scalp, e.g., locations above ears, temples, and eyebrows. The first sensor unit 111 may include a micro translucent sensor. The first sensor unit 111 may sense an EEG in time series order, generate a first sensing signal S1, and provide the first sensing signal S1 to the learning unit 131 or the determination unit 133 to be described later.

The second wearable member B2 may be a member that may be worn on a user's body at a different position from the first wearable member B1. The second wearable member B2 may have a structure to be wearable at a position capable of detecting a biometric signal other than an EEG, e.g., an electrocardiogram (ECG) measuring position, an electrooculogram (EOG) measuring position, and an EMG measuring position. The second wearable member B2 may include at least one of a wearable part 2-1 B2-1 that may be worn on the head of a user, a wearable part 2-2 B2-2 that may be worn on a wrist of the user, and a wearable part 2-3 B2-3 that may be worn on the chest of a user. The wearing part 2-1 B2-1 may be integrally connected to the first wearable member B1 worn on the head of a user, but is not necessarily limited thereto. In another embodiment, the second wearable member B2 may include a wearable part 2-4 B2-4 that may be worn on a finger of a user.

The second sensor unit 112 is provided at the second wearable member B2 and may generate a second sensing signal S2 by sensing an EEG and other biometric signals. The second sensor unit 112 may detect at least any one of an EMG, an EOG, an ECG, and a PPG and generate the second sensing signal S2. The second sensor unit 112 may include a sensor 2-1112-1 for detecting an EOG or a PPG, a sensor 2-2-112-2 for detecting an EMG, and a sensor 2-3112-3 for detecting an ECG. Alternatively, the second sensor unit 112 may further include a sensor 2-4112-4 for detecting a PPG.

The sensor 2-1112-1 may be disposed at the wearing part 2-1 B2-1, the sensor 2-2-112-2 may be disposed at wearable part 2-2 B2-2, and the sensor 2-3112-3 may be disposed at the wearing part 2-3 B2-3. Alternatively, the sensor 2-4112-4 may be disposed at the wearing part 2-4 B2-4. However, the present disclosure is not necessarily limited thereto, and the second 2-3112-3 for measuring an ECG may be disposed at the wearing part 2-2 B2-2 worn on a wrist or the wearable part 2-4 B2-4 worn on a finger.

The stimulation means 114 is disposed at the first wearable member B1 and may apply stimulation to the brain according to a stimulation signal provided from the outside. The stimulation means 114 may be an ultrasonic stimulation means for generating ultrasound stimulation. The stimulation means 114 may generate and apply different types of stimulations according to target locations of the brain to apply stimulation. For example, the stimulation means 114 may stimulate a cortical region such as the dorsolateral prefrontal cortex (DLPFC) by using repetitive transcranial magnetic stimulation (rTMS) and stimulate a subcortical region such as the thalamus by using transcranial ultrasound stimulation (TUS). In another embodiment, the stimulation means 114 may be movably coupled to the first wearable member B1. For example, the stimulation means 114 may include a separate driving means to change a physical position to a brain stimulation target position in the first wearable member B1. At this time, a guide rail for guiding the movement of the stimulation means 114 may be installed on the first wearable member B1.

The first communication unit 115 performs a function of transmitting the first sensing signal S1 or the second sensing signal S2 generated by the first sensor unit 111 or the second sensor unit 112 to the server unit 130 and a function of receiving a stimulation signal generated by the determination unit 133 of the server unit 130. The wearable device 110 may directly transmit/receive data to/from the server unit 130 through the first communication unit 115, but may also transmit data to the server unit 130 through the user terminal 20. The first communication unit 115 may include a communication means capable of communicating with the user terminal 20, e.g., Bluetooth, ZigBee, Medical Implant Communication Service (MISC), and Near Field Communication (NFC).

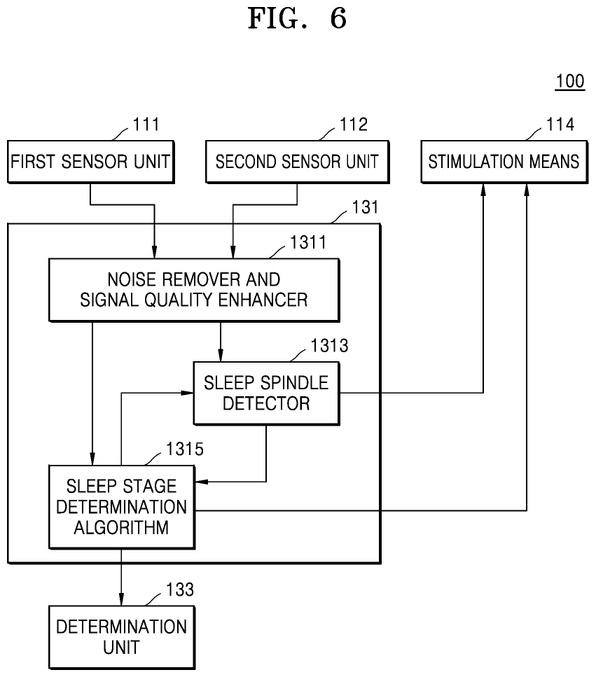

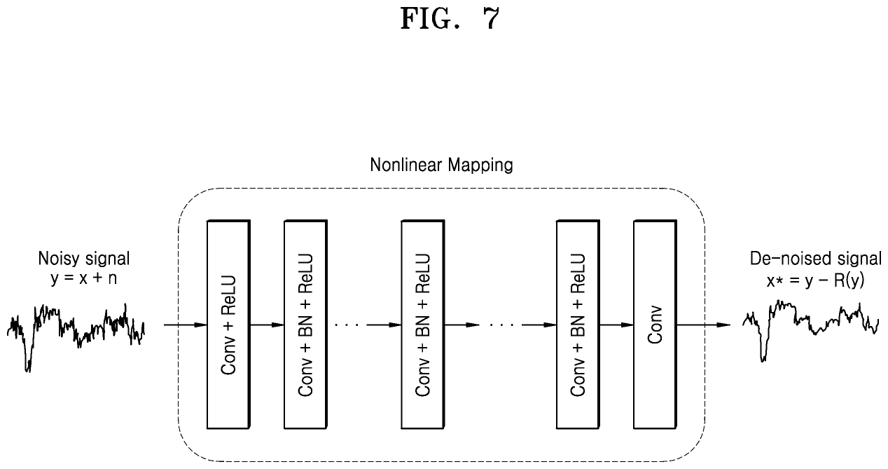

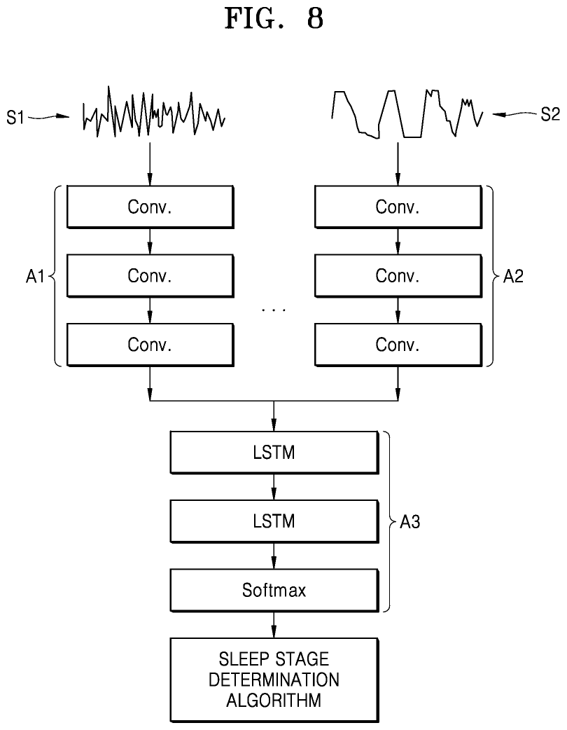

FIG. 6 is a diagram showing a structure for determining a sleep stage and controlling ultrasound stimulation in an artificial intelligence-based noninvasive brain circuit control therapy system for sleep enhancement according to an embodiment of the present disclosure, FIG. 7 is a diagram for describing a noise remover and signal quality enhancer 1311 using a convolutional neural network (CNN), and FIG. 8 is a diagram for describing a sleep stage determination algorithm.

Referring to FIGS. 6 to 8, an artificial intelligence-based noninvasive brain circuit control therapy system for sleep enhancement according to an embodiment of the present disclosure discovers core human brain circuits related to sleep enhancement and determines a sleep stage by using an EEG to perform non-invasive local brain stimulation. At this time, the artificial intelligence-based noninvasive brain circuit control therapy system for sleep enhancement according to an embodiment of the present disclosure generates a determination algorithm for determining a sleep stage by using an EEG based on deep learning and determine a sleep stage based on the generated determination algorithm.

Here, the server unit 130 may correspond to at least one processor or may include at least one processor. Therefore, the server unit 130 may be driven while being included in a hardware device, such as a microprocessor or general purpose computer system. Here, the ‘processor’ may refer to, for example, a data processing device embedded in hardware, having circuitry physically structured to perform functions represented by code or instructions in a program. Examples of the data processing device embedded in hardware may include a microprocessor, a central processing unit (CPU), a processor core, a multiprocessor, an application-specific integrated circuit (ASIC), and a field programmable gate array (FPGA), but the present disclosure is not limited thereto.

The server unit 130 may include a learning unit 131, a determination unit 133, and a second communication unit 135. However, this is merely one embodiment, and the learning unit 131 and the determination unit 133 may not be arranged in one server unit 130. In other words, the learning unit 131 may be disposed in the server unit 130, and the determination unit 133 may be disposed in the user terminal 20 and receive a sleep stage determination algorithm generated by the learning unit 131 to determine a sleep stage. Also, in another embodiment, both the learning unit 131 and the determination unit 133 may be disposed in the user terminal 20. Hereinafter, for convenience of explanation, a case in which the learning unit 131 and the determination unit 133 are provided in one server unit 130 will be mainly described.

The learning unit 131 may machine-learn a criterion for determination of a sleep stage of a user based on the first sensing signal S1 generated by the first sensor unit 111 and the second sensing signal S2 generated by the second sensor unit 112. The learning unit 131 learns the criterion for determination based on deep learning, wherein the deep learning is defined as a set of machine learning algorithms that attempt high-level abstraction (a task of summarizing key contents or functions in a large amount of data or complex data) through a combination of various non-linear transformation methods. The learning unit 131 may use any one of deep learning models including, for example, a deep neural network (DNN), a convolutional neural network (CNN), a recurrent neural network (RNN), and a deep belief network (DBN).

The learning unit 131 may use algorithms and/or methods (techniques) such as linear regression, regression tree, kernel regression, support vector regression, and deep learning to predict a sleep stage or generate appropriate ultrasound stimulation.

Also, the learning unit 131 may use algorithms and/or methods (techniques) such as principal component analysis, non-negative matrix factorization, independent component analysis, manifold learning, and SVD for vector calculation.

The learning unit 131 may use algorithms and/or methods (techniques) such as k-means, hierarchical clustering, mean-shift, and self-organizing maps (SOMs) for grouping information.

The learning unit 131 may use algorithms and/or methods (techniques) such as bipartite cross-matching, n-point correlation two-sample testing, and minimum spanning tree for data comparison.

The learning unit 131 may machine-learn by using the first sensing signal S1 generated by the first sensor unit 111 by detecting EEGs in time series order and a second sensing signal S2 generated by the second sensor unit 112 by detecting other biometric signals in time series order. The learning unit 131 may extract a first feature from the first sensing signal S1, extract a second feature from the second sensing signal S2, and, based on the first feature and the second feature, learn a criterion for determination. Meanwhile, the learning unit 131 may store a general common criterion for determination of a sleep stage of a person in advance and may learn a criterion for determination based on the common criterion for determination and a first feature and a second feature extracted from a particular user. Through this, the learning unit 131 may generate a user-customized criterion for determination through deep learning based on the common criterion for determination.

At this time, when the first sensing signal S1 and the second sensing signal S2 are provided, the learning unit 131 may remove noise and amplify the signal quality through the noise remover and signal quality enhancer 1311. The learning unit 131 may generate a learning data signal y by adding a random noise n to an actual EEG x of a user prior to the noise removal by the noise remover and signal quality enhancer 1311 and output R(y) by applying residual learning to the learning data signal y. The learning unit 131 learns parameters of a network to reduce a difference between the output R(y) of the network and the noise n during a learning process. In this case, a signal from which the final noise is removed may be obtained as follows.

x*=y−R(y)

A convolution layer and a rectified linear unit (Relu) of FIG. 7 are convolutional measurement and nonlinear operation layers and are hierarchically configured as shown in FIG. 7. In detail, as shown in FIG. 7, a filter having a size of 3*3*1 may be used to generate 64 feature maps in a first layer, and an activation function may be included. The activation function may be applied to each layer to perform a function of making inputs to have a complex non-linear relationship therebetween. As the activation function, a Sigmoid function capable of converting an input into a normalized output, a tanh function, a ReLU, a Leacky ReLU, etc. may be used. In the present disclosure, a case of using the ReLU will be mainly described. In an embodiment, the learning unit 131 performing learning by using 64 filters having a size of 3*3*64 for second to seventeenth layers, adding a batch normalization layer between a convolution layer and a ReLU, and using one filter having a size of 3*3*64 for the last layer to generate an output signal from which noise is removed.

Meanwhile, the noise remover and signal quality enhancer 1311 may use an algorithm for increasing a sampling rate as an example of preprocessing for signal quality amplification. In other words, a sleep signal obtained at 100 Hz may be amplified to 200 Hz through upsampling and used. In this case, a control unit learns network parameters by modifying the learning data signal y as follows.

y=U(D(x))

Here, the function D(x) is a down-sampling function, and the function U(x) is an up-sampling function.

The learning unit 131 may generate a sleep signal by removing noise and amplifying the signal quality from an actually sensed EEG by using the noise remover and signal quality enhancer 1311 trained through the above process. Of course, the above-stated process may be applied not only to an EEG, but also to other biometric signals other than an EEG. The learning unit 131 may receive the sleep signal and output at least one of wake, sleep, and sleep stages N, N2, N3, and REM according to the criterion for determination, which is a sleep stage determination algorithm.

In another embodiment, the learning unit 131 may learn the criterion for determination by using the sleep signal, but may also detect sleep spindles from a sleep signal through a sleep spindle detector 1313 and learns the criterion for determination by using the sleep spindles. Sleep spindles may be found by a detector that detects a neural oscillatory activity with a frequency from 10 Hz to 16 Hz lasting for at least 0.5 seconds during continuous measurement of a multi-biometric signal on the system through an artificial intelligence algorithm in real time. The sleep spindles detected in this way may be delivered together with a sleep stage through an external device, which is a mobile device, and a pre-set ultrasound stimulation is applied to the thalamus, the anterior cingulated cortex, the subcallosal cingulated cortex, the hippocampus, and the basal forebrain/medial frontal cortex, which are known as brain regions for cognitive-emotional control, by using the sleep spindles and the sleep stage.

A neural network structure used thereafter in the process that the learning unit 131 learns the criterion for determination may be divided into two processes A1 and A3. In detail, the learning unit 131 may learn a filter to extract features from an EEG through one channel in a first process A1. The first process A1 may use a convolutional neural network (CNN). The learning unit 131 may set different filter kernel sizes for respective convolutional neural networks, such that a small filter size convolutional neural network may detect temporary changes in signals and a large filter size convolutional neural network may capture longer-term signal fluctuations.

The learning unit 131 may learn a criterion for determination by using not only the first sensing signal S1 generated by sensing an EEG, but also the second sensing signal S2 generated by sensing other biometric signals. In this case, the first sensing signal S1 and the second sensing signal S2 may each be subjected to an indirect learning process to extract features. A filter to extract features from an EEG may be learned in the first process A1, and a filter to extract features from other biometric signals may be learned in the second process A2. The first process A1 and the second process A2 may each be configured as a CNN and may each have a multi-channel neural network structure. For example, when two CNN channels are used, an EEG and an ECG may be input thereto, respectively.

The learning unit 131 may learn to encode temporal information such as a transition rule of a sleep stage from a first feature or from a first feature and a second feature extracted in a previous stage through a third process A3. The learning unit 131 may include two bidirectional long short term memory (B-LSTM) layers, and temporal information may be added to the first feature and the second feature learned from the first process A1 and the second process A2 through a short connection.

On the other hand, referring again to FIG. 4, the determination unit 133 may determine a current sleep stage of a user by using the criterion for determination generated by the learning unit 131 and a measured multi-biometric signal, generate a stimulation signal corresponding to the determined current sleep stage, and provide the stimulation signal to a stimulation means.

In the determination unit 133, a criterion for determination of a sleep stage generated by the learning unit 131 may be stored in advance. The determination unit 133 may determine a current sleep stage of a user according to a first sensing signal and a second sensing signal provided from the wearable device 110 by the criterion for determination.

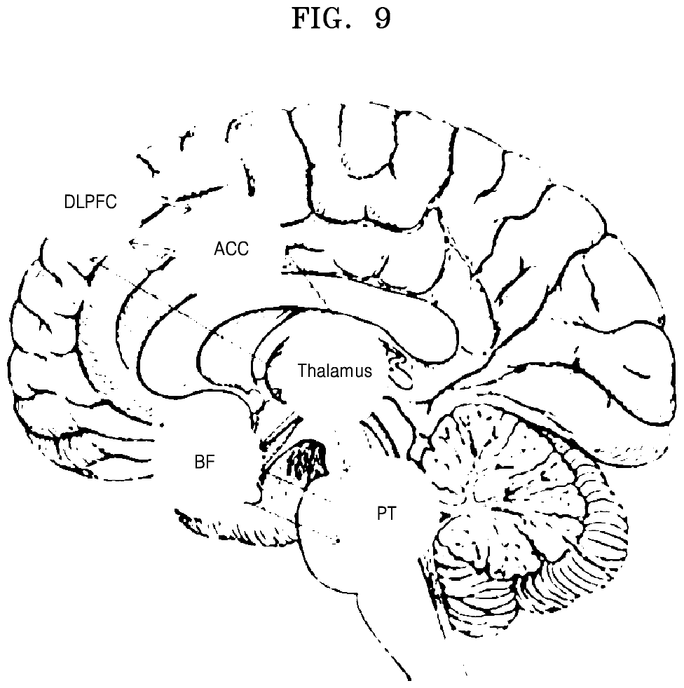

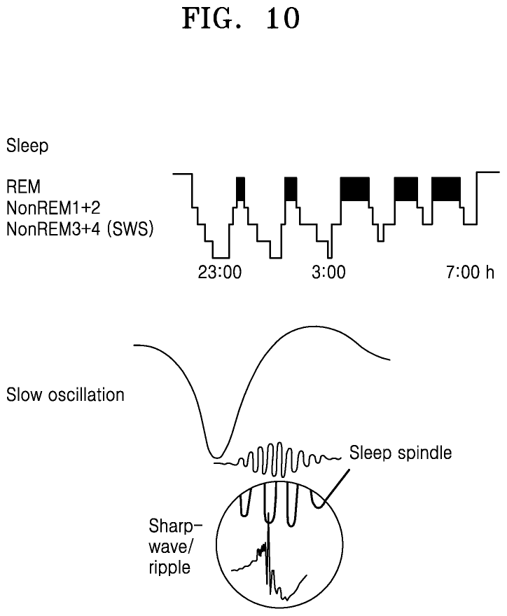

Also, when the current sleep stage of the user is determined as described above, the determination unit 133 may generate a stimulation signal corresponding to the sleep stage according to a pre-set purpose. More detailed descriptions thereof will be given below with reference to FIGS. 9 and 10.

FIG. 9 is a diagram showing major parts of the human brain for controlling sleep, and FIG. 10 is a diagram showing a time distribution of each stage of REM sleep and NREM sleep during overnight sleep and a correlation between sleep spindles and slow wave and high frequency EEG.

Referring to FIGS. 9 and 10, in an embodiment, an artificial intelligence-based noninvasive brain circuit control therapy system for sleep enhancement according to an embodiment of the present disclosure may induce effective sleep initiation and emotional relaxation in a wake phase through ultrasonic stimulation. In other words, the determination unit 133 according to an embodiment of the present disclosure may detect a multi-biometric signal including an EEG of a user and, when it is detected from the multi-biometric signal by a sleep stage determining algorithm that the alpha wave continues in the EEG of the user at the beginning of sleep, may apply ultrasonic stimulation by using the stimulation means 114 of the wearable device 110 to effectively induce sleep. Here, the above-mentioned brain regions may be the dorsolateral prefrontal cortex (DLPFC) and anterior cingulated cortex (ACC) regions, which are known to relieve tension and have an anti-anxiety effect. The determination unit 133 may control a stimulation apparatus to apply ultrasonic stimulation to the brain regions to induce NREM sleep.

In another embodiment, to enhance hippocampal memory, the determination unit 133 may apply ultrasound stimulation similar to spindles to the thalamus and the basal forebrain to activate sleep spindles and hippocampal neurons when a slow wave sleep stage is detected from an EEG.

Alternatively, after determining a sleep stage by an artificial intelligence, the determination unit 133 may be implemented to automatically stimulate different brain regions by matching brain stimulation parameters suitable for the respective brain regions for sleep interruption needed for each situation. When NREM stage 2 sleep spindles are detected, the determination unit 133 may connect thalamoreticular nucleus stimulation to enhance thalamocortical oscillation to enhance slow wave sleep or, in the slow wave sleep stage, connect thalamoreticular nucleus stimulation and a stimulation for activating a brain circuit connected to the hippocampus of the medial temporal lobe. Also, when REM sleep is detected, the determination unit 133 may stimulate the cingulated cortex to enhance not only the thalamoreticular nucleus but also the emotion regulation mechanism in REM sleep.

While the biological clock of the hypothalamus adjusts the wake-sleep state according to the day-night cycle, in the case of night shift workers or shift workers, it is necessary to maintain the wake state and increase concentration in a dark environment at night. To activate the night shift mode in the environment with reduced light stimulation to enhance the wake state and concentration, an algorithm that issues an activation command to apply brain stimulation to the basal forebrain bundle may be employed.

As described above, an artificial intelligence-based noninvasive brain circuit control therapy system for sleep enhancement according to embodiments of the present disclosure not only recognizes a sleep state of a user through analysis of a multi-biometric signal, but also appropriately determines the surrounding environment or various situations to match various neuromodulatory stimulation modes for inducing cognitive-emotional regulation and enhancements while being in an appropriate sleep-wake state according to the surrounding environment and a corresponding situation.

The embodiments according to the present disclosure described above may be implemented in the form of a computer program that may be executed through various components on a computer, and such a computer program may be recorded on a computer-readable medium. At this time, the medium may be to store a program executable by a computer. Examples of the medium include magnetic media, such as a hard disk, a floppy disk, and a magnetic tape, optical recording media, such as CD-ROM and DVD, magneto-optical media such as a floptical disk, and ROM, RAM, and a flash memory, which are configured to store program instructions.

Meanwhile, the computer program may be specially designed and configured for the present disclosure or may be known and available to one of ordinary skill in the computer software field. Examples of computer programs may include machine language code such as code generated by a compiler, as well as high-level language code that may be executed by a computer using an interpreter or the like.

While the present disclosure has been described with reference to exemplary embodiments, it is to be understood that the present disclosure is not limited to the disclosed example embodiments, but, on the contrary, is intended to cover various modifications and equivalent arrangements included within the spirit and scope of the appended claims. Accordingly, the true scope of protection of the present disclosure should be determined by the technical idea of the appended claims.

### INDUSTRIAL APPLICABILITY

According to an embodiment of the present disclosure, there are provided an artificial intelligence-based noninvasive brain circuit control therapy system for sleep enhancement and a method therefor. Also, embodiments of the present disclosure may be applied to industrial noninvasive brain circuit control.

1. An artificial intelligence-based noninvasive brain circuit control therapy system for sleep enhancement, the system comprising:
a wearable device comprising a first wearable member and a second wearable member formed to be wearable on a body of a user, a first sensor unit disposed on the first wearable member to detect an electroencephalogram (EEG), a second sensor unit disposed on the second wearable member to detect a biometric signal different from the EEG, and a stimulation means disposed on the first wearable member to stimulate the brain according to a stimulation signal provided thereto;
a learning unit configured to machine-learn a criterion for determination of a sleep stage of the user based on a first sensing signal generated by the first sensor unit and a second sensing signal generated by the second sensor unit; and
a determination unit configured to determine a current sleep stage of the user based on the criterion for determination, generate a stimulation signal corresponding to a determined sleep stage, and provide the stimulation signal to the stimulation means.

2. The system of claim 1, wherein
the second sensor unit detects an electrooculogram (EOG) and generates the second sensing signal, and
the second wearable member is connected to the first wearable member and is wearable on the head of the user.

3. The system of claim 1, wherein
the second sensor unit detects an electromyogram (EMG) and generates the second sensing signal, and
the second wearable member is wearable on a wrist of the user or is connected to the first wearable member and wearable on the face of the user.

4. The system of claim 1, wherein
the second sensor unit detects a photoplethysmogram (PPG) and generates the second sensing signal, and
the second wearable member is wearable on the chest or a finger of the user or is connected to the first wearable member and wearable on an ear of the user.

5. The system of claim 1, wherein
the second sensor unit generates the second sensing signal by sensing an EOG, an EMG, and a PPG, and
the second wearable member comprises a wearable part 2-1 that is connected to the first wearable member and is wearable on the head of the user, a wearable part 2-2 that is wearable on a wrist of the user, and a wearable part 2-3 that is wearable on the chest of the user.

6. The system of claim 1, wherein the stimulation means is an ultrasound generating means for generating ultrasound stimulation.

7. The system of claim 1, wherein
the first sensor unit generates the first sensing signal by sensing the EEG in the time series order, and
the second sensor unit generates the second sensing signal synchronized with the first sensing signal by detecting the other biometric signal in time series order.

8. The system of claim 7, wherein the learning unit extracts a first feature from the first sensing signal generated in the time series order, extracts a second feature from the second sensing signal generated in the time series order, and learns the criterion for determination based on the first feature and the second feature including temporal information.

9. An artificial intelligence-based noninvasive brain circuit control therapy method for sleep enhancement, the method comprising:
receiving a first sensing signal generated by a first sensor unit that detects an electroencephalogram (EEG);
receiving a second sensing signal generated by a second sensor unit that detects a biometric signal other than the EEG; and
machine-learning a criterion for determination of a sleep stage of a user based on the first sensing signal and the second sensing signal.

10. The method of claim 9, wherein
the first sensor unit generates the first sensing signal by sensing the EEG in the time series order, and
the second sensor unit generates the second sensing signal synchronized with the first sensing signal by detecting the other biometric signal in time series order.

11. The method of claim 10, wherein the machine-learning of the criterion for determination comprises:
extracting a first feature from the first sensing signal generated in the time series order;
extracting a second feature from the second sensing signal generated in the time series order; and
learning the criterion for determination based on the first feature and the second feature including temporal information.

12. The method of claim 11, wherein the extracting of the first feature and the extracting of the second feature are performed noninvasively.

13. The method of claim 9, further comprising:
determining a current sleep stage of the user based on the criterion for determination; and
generating a stimulation signal corresponding to a determined sleep stage and providing the stimulation signal to a stimulation means.

14. The method of claim 9, wherein the second sensor unit detects an electrooculogram (EOG) and generates the second sensing signal.

15. The method of claim 9, wherein the second sensor unit detects an electromyogram (EMG) and generates the second sensing signal.

16. The method of claim 9, wherein the second sensor unit detects a photoplethysmogram (PPG) and generates the second sensing signal.

17. The method of claim 9, wherein the second sensor unit detects an EOG, an EMG, and a PPG to generate the second sensing signal.

18. A computer program stored in a medium for executing the method of claim 9 by using a computer.
